(19) United States
(12) Patent Application Publication
(10) Pub. No.: US 2017/0054079 A1
(43) Pub. Date: Feb. 23, 2017

(54) IODINATION PROCESS, MONOMER AND POLYMER

(71) Applicants: Cambridge Display Technology Limited, Godmanchester (GB); Sumitomo Chemical Company Limited, Tokyo (JP)

(72) Inventors: Florence Bourcet, Godmanchester (GB); Annette Steudel, Dresden (DE)

(21) Appl. No.: 15/301,261

(22) PCT Filed: Mar. 30, 2015
(86) PCT No.: PCT/GB2015/050970

§ 371 Date: Sep. 30, 2016

(30) Foreign Application Priority Data
Mar. 31, 2014 (GB) 1405771.5

(51) Int. Cl. H01L 51/00; C09K 11/02; C07D 409/14; C08G 61/12; C07D 333/76

(52) U.S. Cl. CPC H01L 51/0036; C08G 61/126; C07D 333/76; C07D 409/14; C08G 61/124; H01L 51/0043; C09K 11/025; C08G 2261/12; C08G 2261/3241; C08G 2261/3243; C08G 2261/1412; C08G 2261/18; C08G 2261/312; C08G 2261/95; H01L 51/5016

(57) ABSTRACT

The present invention relates to processes for the preparation of iodinated compounds of formula (I): (Formula (I)) wherein R¹ is the same or different in each occurrence and is a substituent; m independently in each occurrence is 0, 1, 2 or 3; and X independently in each occurrence is NR², PR², —CR²₂—, —SiR²₂, O or S wherein R² is the same or different in each occurrence and is a substituent.

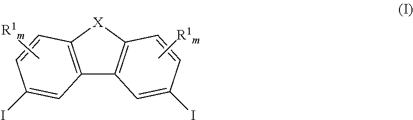

### BACKGROUND OF THE INVENTION

Electronic devices containing active organic materials are attracting increasing attention for use in devices such as organic light emitting diodes (OLEDs), organic photoresponsive devices (in particular organic photovoltaic devices and organic photosensors), organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An OLED has a substrate carrying an anode, a cathode and one or more organic light-emitting layers between the anode and cathode.

A light emitting layer may comprise a semiconducting host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton).

Phosphorescent dopants are also known (that is, a light-emitting dopant in which light is emitted via decay of a triplet exciton).

Sook et al., J. Mater. Chem., 2011, 21, 14604-14609, discloses formation of hosts DBT1, DBT2 and DBT3 from dibromodibenzothiophene:

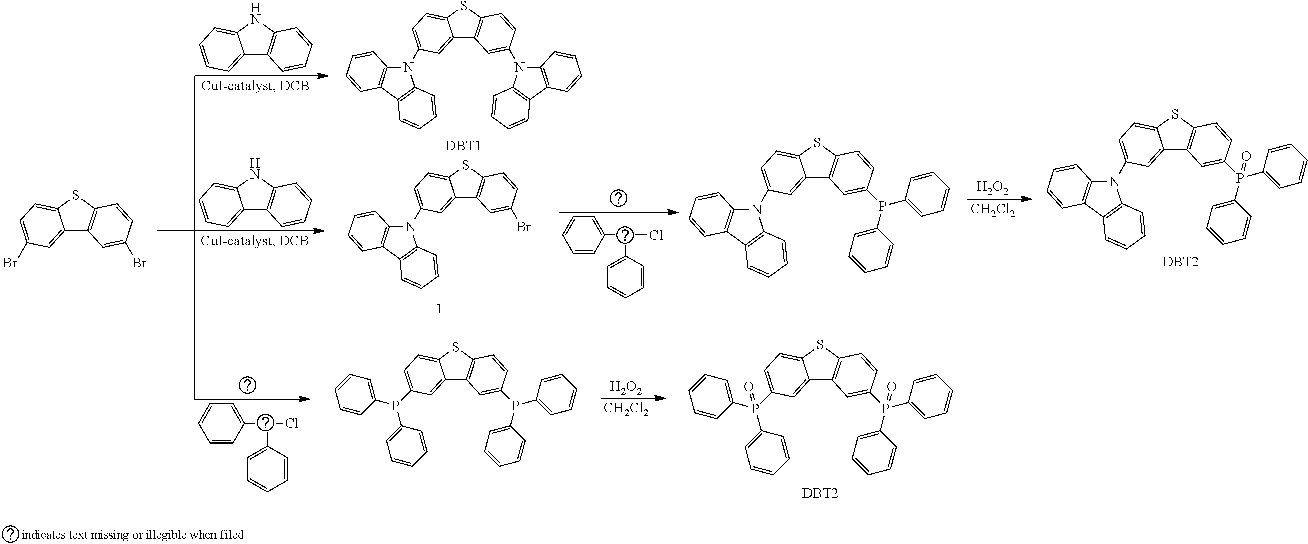

The preparation of 2,8-diiododibenzothiophene has been described in Journal of Organic Chemistry, 2007, 72, pp 6672-6679, Lo et al. in which dibenzothiophene was iodinated directly with iodine, acetic acid, sulfuric acid and periodic acid in chloroform to obtain 2,8-diiododibezonthiophene in 22-50% yield:

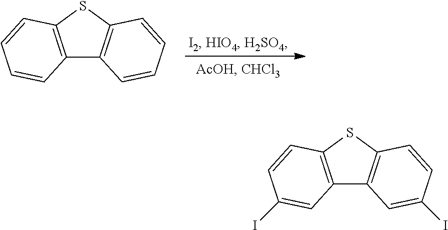 [SMILES: c1ccc2c(c1)sc1ccccc12 ; Ic1ccc2sc3ccc(I)cc3c2c1]

A similar iodination is described in Organic Letters, 2010, vol. 12, pp 2194-2197, Feng et al. which describes the direct iodination of dibenzothiophene and dibenzofuran with iodine, acetic acid, sulfuric acid and periodic acid. The corresponding di-iodo compounds were obtained in 31-66% yield.

A wide variation in yield is reported for the same reaction in both the prior art documents discussed above. In addition there is the potential for unwanted side reactions to occur, for example, by over or under iodination of the aromatic ring or by iodination at different positions on the aromatic ring. In addition the reaction can also produce unwanted oxidation products via oxidation of the sulphur.

On repetition of the prior art processes, the inventor has found the direct iodination process gave only a very small amount of the desired product and poor conversion of the starting material. Furthermore the inventor has found that these processes may require purification by column chromatography which can be difficult to scale up.

### SUMMARY OF THE INVENTION

A first aspect according to the present invention provides a process for the preparation of a compound of formula (I):

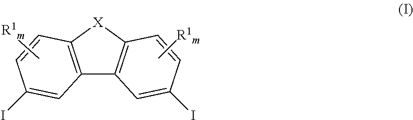

comprising (i) metalation and (ii) iodination of a compound of formula (II):

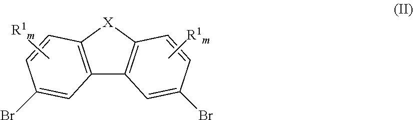

wherein R¹ is the same or different in each occurrence and is a substituent; m independently in each occurrence is 0, 1, 2 or 3; and X independently in each occurrence is NR², PR², —CR²₂—, —SiR²₂, O or S wherein R² is the same or different in each occurrence and is a substituent.

Preferably, m is 0 and X is O or S. Most preferably, m is 0 and X is S.

In a second aspect, the invention provides a process of forming a compound of formula (V):

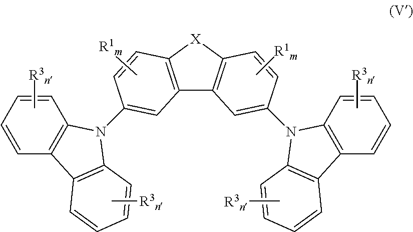

the process comprising the step of reacting a compound of formula (I) and a compound of formula (IV′):

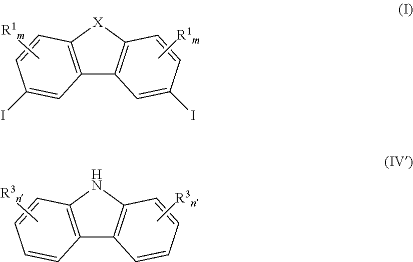

wherein R¹ is the same or different in each occurrence and is a substituent; m independently in each occurrence is 0, 1, 2 or 3; X independently in each occurrence is NR², PR², —CR²₂—, —SiR²₂, O or S wherein R² is the same or different in each occurrence and is a substituent; R³ in each occurrence is a substituent; and n′ in each occurrence is independent 0, 1, 2, 3 or 4, with the proviso that at least one n′ is 1 and at least one R³ is bromine.

R¹, R², m, and X may be as described anywhere herein. Any groups R³ other than the one or more bromine R³ groups may be as described anywhere herein. Optionally, one n′ of formula (IV′) is 1 and the other n′ is 0. such that the compound of formula (V′) carries two bromine substituents R³ and the compound of formula (V′) is suitable for use as a monomer in polymerisation as described anywhere herein.

In a third aspect the invention provides a partially conjugated co-polymer comprising a repeat unit of formula (Vr) and at least one co-repeat unit:

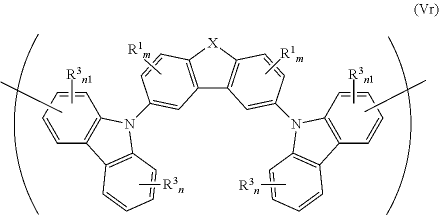

wherein R¹ is the same or different in each occurrence and is a substituent; m independently in each occurrence is 0, 1, 2 or 3; X independently in each occurrence is NR², PR², —CR²₂—, —SiR²₂, O or S wherein R² is the same or different in each occurrence and is a substituent; R³ in each occurrence is a substituent; n in each occurrence is independently 0, 1, 2, 3 or 4; and n1 in each occurrence is independently 0, 1, 2 or 3.

R¹, R², R3², n, n1, m, and X of the third aspect may be as described anywhere herein.

### DESCRIPTION OF THE DRAWINGS

FIG. 1 is an HPLC chromatogram of a sample of the reaction mixture of a process according to the present invention, and

FIG. 2 is an HPLC chromatogram of a sample of the reaction mixture of a literature process using direct iodination.

### DETAILED DESCRIPTION OF THE INVENTION

The invention provides a two step process to obtain compounds of formula (I) reproducibly, in high yield and without the need for purification by column chromatography.

A first aspect according to the present invention provides a process for the preparation of a compound of formula (I):

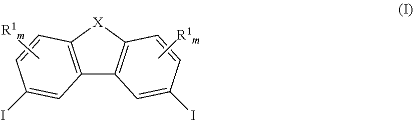

comprising (i) metalation and (ii) iodination of a compound of formula (II):

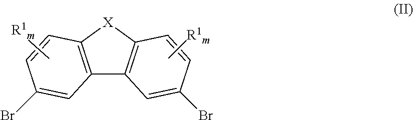

wherein R¹ is the same or different in each occurrence and is a substituent; m independently in each occurrence is 0, 1, 2 or 3; and X independently in each occurrence is NR², PR², —CR²₂—, —SiR²₂, O or S wherein R² is the same or different in each occurrence and is a substituent.

Overall, after the metalation and iodination steps, the bromo substituent in the compound of formula (II) is replaced with an iodo substituent to form the compound of formula (I). The process enables regiospecific introduction of the iodo substituent into the compound of formula (II) to form the compound of formula (I).

Preferably, m is 0.

Each R¹, where present, may independently in each occurrence be selected from:
alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F; and
aryl and heteroaryl groups that may be unsubstituted or substituted with one or more substituents, preferably phenyl substituted with one or more C₁₋₂₀ alkyl groups.

Preferably, R¹ is a C₁₋₄₀ hydrocarbyl group.

Preferably, X is S or O. Preferably, X is O. More preferably, X is S.

Preferably, m is 0 and X is O or S. Most preferably, m is 0 and X is S.

Each R², where present, may independently in each occurrence be selected from:
H;
alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F; and
aryl and heteroaryl groups that may be unsubstituted or substituted with one or more substituents, preferably phenyl substituted with one or more C₁₋₂₀ alkyl groups.

The two groups R² of —CR²₂—, —SiR²₂ may be linked to form a ring.

Preferably, R² is a C₁₋₄₀ hydrocarbyl group.

In a preferred embodiment the metalation is lithiation. Preferably, the lithiation is carried out using an organolithium, more preferably an alkyllithium, such as n-butyllithium or s-butyllithium. Most preferably, the lithiation is carried out using s-butyllithium.

Preferably, the metalation is carried out in an aprotic organic solvent, for example THF.

Preferably, the metalation reaction mixture is maintained at a temperature between −60 and −100° C., more preferably at a temperature of between −70 and −80° C.

The iodination step is carried out subsequently to the metalation step. The metalation step provides a reaction mixture which may contain lithiated reaction intermediates derived from the compound of formula (II). Any reaction intermediates formed after the metalation step remain in situ in the reaction mixture and are used directly in the iodination step.

In a preferred embodiment, the iodination is carried out using elemental iodine. Preferably, the elemental iodine is added directly to the reaction mixture obtained after the metalation.

Preferably, the iodination is commenced at a temperature between −60 and −100° C. and then warmed to room temperature. More preferably, the iodination is commenced at a temperature of between −70 and −80° C. and then warmed to room temperature.

Preferably, after the iodination is complete the compound of formula (I) is obtained.

In a preferred embodiment, the compound of formula (I) obtained is purified by recrystallisation. Preferably, the compound of formula (I) obtained is purified by recrystallisation using toluene.

Preferably, the compound of formula (I) obtained is not purified by chromatography.

Preferably, the compound of formula (I) is obtained in a yield of 70% or more, preferably 80% or more, preferably 90% or more, preferably 95% or more.

Preferably, the compound of formula (I) is obtained with a chemical purity of greater than about 95%; or greater than about 96%, or greater than about 97%, or greater than about 97.3% (as measured by HPLC).

In a preferred embodiment, the compound of formula (II) is prepared by bromination of a compound of formula (III):

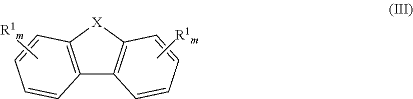

Preferably, the bromination is carried out using elemental bromine.

Preferably, the bromination is carried out in a mixed solvent system comprising a non-polar organic solvent and polar protic solvent. Preferably, the non-polar organic solvent is chloroform or dichloromethane. Preferably, the polar protic solvent is acetic acid. Preferably, the non-polar solvent is chloroform and the polar protic solvent is acetic acid.

In a preferred embodiment, the compound of formula (II) obtained is purified by recrystallisation. Preferably the recrystallisation uses one or more solvents selected from the group consisting of aliphatic esters, aliphatic nitriles and aromatic hydrocarbons. Preferably the aliphatic ester solvent is selected from the group consisting of ethyl acetate, butyl acetate and isopropyl acetate. Most preferably the aliphatic ester solvent is n-butyl acetate. Preferably the aliphatic nitrile solvent is selected from the group consisting of acetonitrile and propionitrile. Most preferably, the aliphatic nitrile solvent is acetonitrile. Preferably the aromatic hydrocarbon solvent is selected from the group consisting of benzene and toluene. Most preferably the aromatic hydrocarbon solvent is toluene. A particularly preferred recrystallisation solvent system is toluene and acetonitrile.

Preferably, the compound of formula (II) obtained is not purified by chromatography.

Preferably, the compound of formula (II) is obtained in a yield of 70% or more, preferably 80% or more, preferably 90% or more, preferably 95% or more.

Preferably, the compound of formula (II) is obtained with a chemical purity of greater than about 95%; or greater than about 96%, or greater than about 97%, or greater than about 98%, or greater than about 99%, or greater than about 99.6% (as measured by HPLC).

In a preferred embodiment, the compound of formula (I) obtained is used in a further step comprising reacting the compound of formula (I) with a compound of formula (IV):

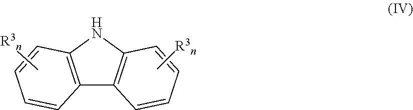

wherein R³ is the same or different in each occurrence and is a substituent; and n independently in each occurrence is 0, 1, 2, 3 or 4.

The reaction of compounds of formulae (I) and (IV) is shown in Scheme 1:

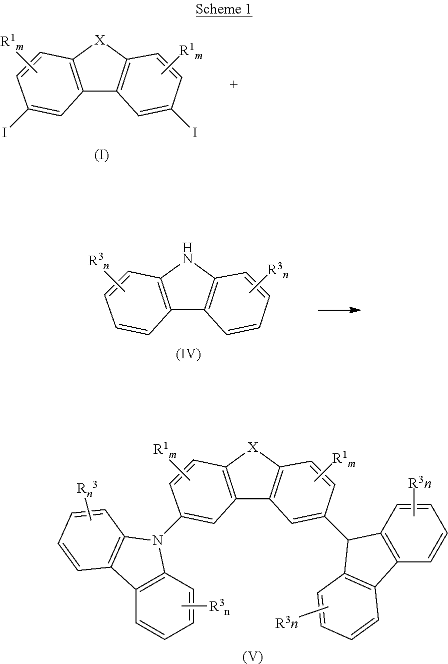

Each R³, where present, may independently in each occurrence be selected from:
a leaving group such as Br, Cl, benzenesulphonyl, p-toluenesulphonyl (tosyl), methylsulphonyl (mesyl), or trifluoromethanesulphonyl (triflate);
alkyl, optionally C₁₋₂₀ alkyl, wherein one or more non-adjacent C atoms may be replaced with optionally substituted aryl or heteroaryl, O, S, substituted N, and one or more H atoms may be replaced with F; and
aryl and heteroaryl groups that may be unsubstituted or substituted with one or more substituents, preferably phenyl substituted with one or more C₁₋₂₀ alkyl groups.

Preferably, R³ is a C₁₋₄₀ hydrocarbyl group.

In one embodiment, each n is 0.

In another embodiment, one or each n is at least 1, and at least one R³ is Br. Generally, when the compound of formula (IV) comprises a bromo substituent this substituent does not react during the reaction of the compound of formula (IV) with the compound of formula (I) because reaction occurs preferentially at the position of the iodo substituents of the compound of formula (I). This preferential reaction enables the preparation of carbazole substituted compounds, such as carbazole substituted dibenzothiophenes and carbazole substituted dibenzofurans, in which the carbazole is substituted with bromo substituents. The compound of formula (V) may be used as a monomer in a polymerisation in the case where at least two R³ groups of formula (V) are bromine. The compound of formula (V) may be used as a polymer end-capping group in the case where one R³ group is bromine.

In a preferred embodiment in the case where n is at least 1, only one n of formula (IV) is 1 and R³ is Br.

Preferably, at least two equivalents of the compound of formula (IV) are reacted with one equivalent of the compound of formula (I). i.e. the stoichiometric ratio of the compound of formula (IV) to the compound of formula (I) is at least 2 to 1.

Preferably, the compound of formula (IV) is bromocarbazole (IVa):

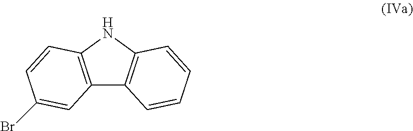 [SMILES: Brc1ccc2[nH]c3ccccc3c2c1]

Preferably, the compound of formula (IVa) is reacted with the compound of formula (I) to form a compound of formula (Va):

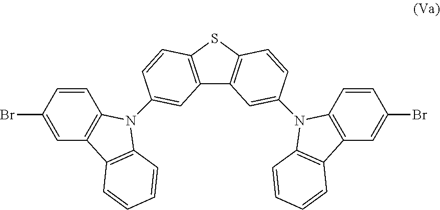 [SMILES: Brc1ccc2c(c1)c1ccccc1n2-c1ccc2sc3ccc(-n4c5ccccc5c5cc(Br)ccc54)cc3c2c1]

The compound of formula (Va) is a specific example of compounds of formula (V).

In a preferred embodiment, the compound of formula (V) obtained is purified by recrystallisation. Preferably the recrystallisation uses one or more solvents selected from the group consisting of alcohols and aromatic hydrocarbons. Preferably the alcohol solvent is selected from the group consisting of methanol, ethanol and isopropanol. Most preferably the alcohol solvent is methanol. Preferably the aromatic hydrocarbon solvent is selected from the group consisting of benzene and toluene. Most preferably the aromatic hydrocarbon solvent is toluene. A particularly preferred recrystallisation solvent system is toluene and methanol.

Preferably, the compound of formula (V) obtained is not purified by chromatography.

Preferably, the compound of formula (V) is obtained in a yield of 70% or more, preferably 80% or more, preferably 90% or more, preferably 95% or more.

Preferably, the compound of formula (V) is obtained with a chemical purity of greater than about 90%; or greater than about 93%, or greater than about 95% (as measured by HPLC).

Polymer Synthesis

The compound of formula (V) may be used to form a conjugated or partially conjugated polymer.

One method of forming conjugated or partially conjugated polymers is Suzuki polymerisation, for example as described in WO 00/53656 or U.S. Pat. No. 5,777,070 which allows formation of C—C bonds between two aromatic or heteroaromatic groups, and so is enables formation of polymers having conjugation extending across two or more repeat units. Suzuki polymerisation takes place in the presence of a palladium complex catalyst and a base.

As illustrated in Scheme 2, in the Suzuki polymerisation process a monomer for forming repeat units RU₁ having leaving groups LG₁ such as boronic acid or boronic ester groups undergoes polymerisation with a monomer for forming repeat units RU₂ having leaving groups LG₂ such as halogen, sulfonic acid or sulfonic ester to form a carbon-carbon bond between Arylene 1 and Arylene 2:

n LG1-RU1-LG1+n LG2-RU2-LG2→-(RU1-RU2)ₙ-

Scheme 2

Exemplary boronic esters have formula (VI):

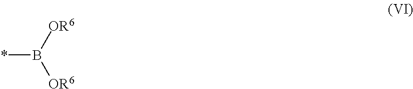

wherein R⁶ in each occurrence is independently a C₁₋₂₀ alkyl group, * represents the point of attachment of the boronic ester to an aromatic ring of the monomer, and the two groups R⁶ may be linked to form a ring. In a preferred embodiment, the two groups R⁶ are linked to form the pinacol ester of boronic acid:

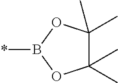 [SMILES: *B1OC(C)(C)C(C)(C)O1]

It will be understood by the skilled person that a monomer LG1-RU1-LG1 will not polymerise to form a direct carbon-carbon bond with another monomer LG1-RU1-LG1. A monomer LG2-RU2-LG2 will not polymerise to form a direct carbon-carbon bond with another monomer LG2-RU2-LG2.

Preferably, one of LG1 and LG2 is bromine or iodine and the other is a boronic acid or boronic ester.

This selectivity means that the ordering of repeat units in the polymer backbone can be controlled such that all or substantially all RU1 repeat units formed by polymerisation of LG1-RU1-LG1 are adjacent, on both sides, to RU2 repeat units.

In the example of Scheme 2 above, an AB copolymer is formed by copolymerisation of two monomers in a 1:1 ratio, however it will be appreciated that more than two or more than two monomers may be used in the polymerisation, and any ratio of monomers may be used.

The base may be an organic or inorganic base. Exemplary organic bases include tetra-alkylammonium hydroxides, carbonates and bicarbonates. Exemplary inorganic bases include metal (for example alkali or alkali earth) hydroxides, carbonates and bicarbonates.

The palladium complex catalyst may be a palladium (o) or palladium (II) compound.

Particularly preferred catalysts are tetrakis(triphenylphosphine)palladium (o) and palladium (II) acetate mixed with a phosphine.

A phosphine may be provided, either as a ligand of the palladium compound catalyst or as a separate compound added to the polymerisation mixture. Exemplary phosphines include triarylphosphines, for example triphenylphosphines wherein each phenyl may independently be unsubstituted or substituted with one or more substituents, for example one or more C₁₋₅ alkyl or C₁₋₅ alkoxy groups.

Particularly preferred are triphenylphospine and tris(ortho-methoxytriphenyl) phospine.

A further polymerisation method is Yamamoto polymerisation in which monomers carrying halogen (preferably bromine) leaving groups react in the presence of a nickel catalyst.

The polymerisation reaction may take place in a single organic liquid phase in which all components of the reaction mixture are soluble. The reaction may take place in a two-phase aqueous-organic system, in which case a phase transfer agent may be used. The reaction may take place in an emulsion formed by mixing a two-phase aqueous-organic system with an emulsifier.

The polymer may be end-capped by addition of an endcapping reactant. Suitable end-capping reactants are aromatic or heteroaromatic materials substituted with only one leaving group. The end-capping reactants may include reactants substituted with a halogen for reaction with a boronic acid or boronic ester group at a polymer chain end, and reactants substituted with a boronic acid or boronic ester for reaction with a halogen at a polymer chain end. Exemplary end-capping reactants are halobenzenes, for example bromobenzene, and phenylboronic acid. End-capping reactants may be added during or at the end of the polymerisation reaction.

Co-Polymers

Polymers formed by reaction of a monomer of formula (V) are preferably copolymers comprising a repeat unit of formula (Vr) and one or more co-repeat units:

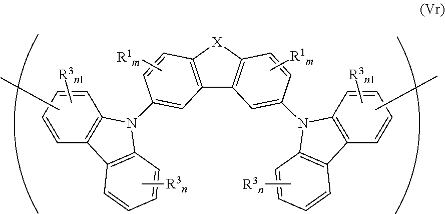

wherein R¹, R³, n, m and X are as described above, and ni in each occurrence is 0, 1, 2 or 3.

The repeat unit of formula (Vr) may have the following formula:

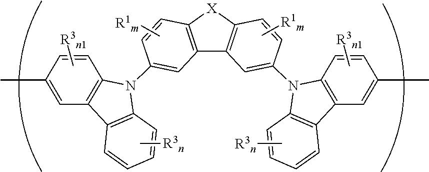

A polymer comprising a repeat unit of formula (Vr) may be a host for a phosphorescent dopant. A composition comprising a polymer comprising a repeat unit of formula (Vr) and a phosphorescent dopant may contain the phosphorescent dopant in an amount in the range of 0.1-50 wt %, optionally 0.5-30 wt %.

If the polymer is a copolymer comprising repeat units of formula (Vr) and one or more co-repeat units then repeat units of formula (Vr) may make up 1-99 mol % of the repeat units of the copolymer, optionally 1-50 mol % of the repeat units.

Preferably, the polymer is a conjugated polymer wherein repeat units in the polymer backbone are conjugated to one another to form a conjugated backbone. The singlet excited state energy level and/or triplet excited state energy level of the polymer may is be controlled by controlling the extent of conjugation of the polymer.

The copolymer may be a partially conjugated copolymer. Repeat units may be introduced into the polymer backbone to limit the extent of conjugation along the polymer backbone and increase an excited stated energy level of the polymer. A partially conjugated polymer may comprise repeat units that limit the extent of conjugation across the repeat unit or that completely break conjugation across the repeat unit.

Repeat units to limit the extent of conjugation along the polymer backbone may be provided in the polymer in an amount in the range of 1-99 mol %, optionally 1-50 mol %.

Substituents may be provided adjacent to one or both linking positions of an arylene co-repeat unit to create steric hindrance with adjacent repeat units, resulting in twisting of the arylene co-repeat unit out of the plane of the adjacent repeat unit.

A twisting repeat unit may have formula (IX):

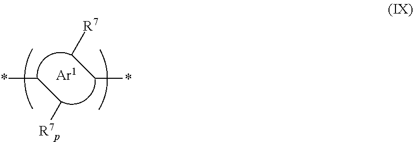

wherein Ar¹ is an arylene group; R⁷ in each occurrence is a substituent adjacent to a linking position of the repeat unit; and p is 0 or 1. The one or two substituents R⁷ may be the only substituents of repeat units of formula (IX), or one or more further substituents may be present, optionally one or more C₁₋₄₀ hydrocarbyl groups.

The one or two substituents R⁷ adjacent to the linking positions of formula (IX) create steric hindrance with one or both repeat units adjacent to the repeat unit of formula (IX).

Each R⁷ may independently be selected from the group consisting of:
alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F;
aryl and heteroaryl groups that may be unsubstituted or substituted with one or more substituents, preferably phenyl substituted with one or more C₁₋₂₀ alkyl groups; and
a linear or branched chain of aryl or heteroaryl groups, each of which groups may independently be substituted, for example a group of formula —(Ar⁷)ᵣ wherein each Ar⁷ is independently an aryl or heteroaryl group and r is at least 2, preferably a branched or linear chain of phenyl groups each of which may be unsubstituted or substituted with one or more C₁₋₂₀ alkyl groups.

In the case where R⁷ comprises an aryl or heteroaryl group, or a linear or branched chain of aryl or heteroaryl groups, the or each aryl or heteroaryl group may be substituted with one or more substituents R⁸ selected from the group consisting of:
alkyl, for example C₁₋₂₀ alkyl, wherein one or more non-adjacent C atoms may be replaced with O, S, substituted N, C═O and —COO— and one or more H atoms of the alkyl group may be replaced with F;
NR⁹₂, OR⁹, SR⁹, SiR⁹₃ and
fluorine, nitro and cyano;

wherein each R⁹ is independently selected from the group consisting of alkyl, preferably C₁₋₂₀ alkyl; and aryl or heteroaryl, preferably phenyl, optionally substituted with one or more C₁₋₂₀ alkyl groups.

Substituted N, where present, may be —NR⁶— wherein R⁶ is a substituent and is optionally in each occurrence a C₁₋₄₀ hydrocarbyl group, optionally a C₁₋₂₀ alkyl group.

Preferably, each R⁷, where present, is independently selected from C₁₋₄₀ hydrocarbyl, and is more preferably selected from C₁₋₂₀ alkyl; unusubstituted phenyl; phenyl substituted with one or more C₁₋₂₀ alkyl groups; and a linear or branched chain of phenyl groups, wherein each phenyl may be unsubstituted or substituted with one or more substituents.

One preferred class of arylene repeat units is phenylene repeat units, such as phenylene repeat units of formula (X):

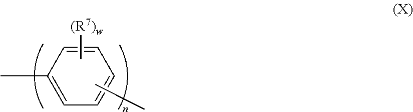

wherein w in each occurrence is independently 0, 1, 2, 3 or 4, optionally 1 or 2; n is 1, 2 or ₃; and R⁷ independently in each occurrence is a substituent as described above.

If n is 1 then exemplary repeat units of formula (X) include the following:

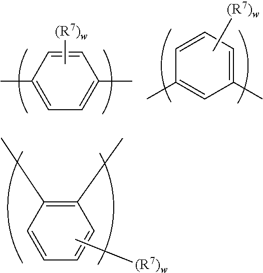

A particularly preferred repeat unit of formula (X) has formula (Xa):

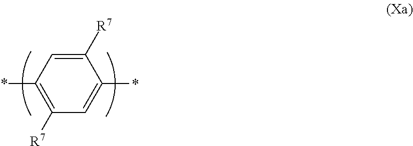

Substituents R⁷ of formula (Xa) are adjacent to linking positions of the repeat unit, which may cause steric hindrance between the repeat unit of formula (Xa) and adjacent repeat units, resulting in the repeat unit of formula (Xa) twisting out of plane relative to one or both adjacent repeat units.

Exemplary repeat units where n is 2 or 3 include the following:

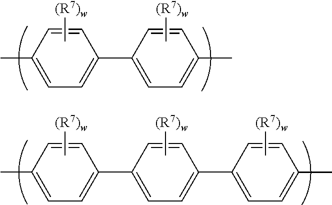

A preferred repeat unit has formula (Xb):

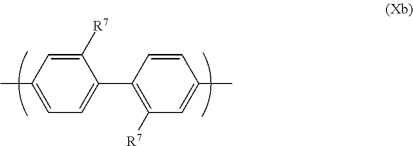

The two R⁷ groups of formula (Xb) may cause steric hindrance between the phenyl rings they are bound to, resulting in twisting of the two phenyl rings relative to one another.

A further class of arylene repeat units is optionally substituted fluorene repeat units, such as repeat units of formula (XI):

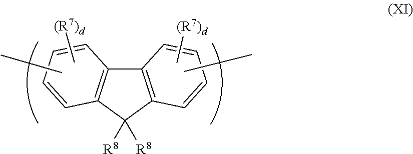

wherein R⁸ in each occurrence is the same or different and is a substituent wherein the two groups R⁸ may be linked to form a ring; R⁷ is a substituent as described above; and d is 0, 1, 2 or 3.

Each R⁸ may independently be selected from the group consisting of:
alkyl, optionally C₁₋₂₀ alkyl, wherein one or more non-adjacent C atoms may be replaced with optionally substituted aryl or heteroaryl, O, S, substituted N, C═O or —COO—, and one or more H atoms may be replaced with F;
aryl and heteroaryl groups that may be unsubstituted or substituted with one or more substituents, preferably phenyl substituted with one or more C₁₋₂₀ alkyl groups; and
a linear or branched chain of aryl or heteroaryl groups, each of which groups may independently be substituted, for example a group of formula —(Ar⁷)ᵣ wherein each Ar is independently an aryl or heteroaryl group and r is at least 2, optionally 2 or 3, preferably a branched or linear chain of phenyl groups each of which may be unsubstituted or substituted with one or more C₁₋₂₀ alkyl groups.

Preferably, each R⁸ is independently a C₁₋₄₀ hydrocarbyl group.

Substituted N, where present, may be —NR⁶— wherein R⁶ is as described above.

The aromatic carbon atoms of the fluorene repeat unit may be unsubstituted, or may be substituted with one or more substituents R⁷ as described with reference to Formula (IX).

Exemplary substituents R⁷ are alkyl, for example C₁₋₂₀ alkyl, wherein one or more non-adjacent C atoms may be replaced with O, S, C═O and —COO—, optionally substituted aryl, optionally substituted heteroaryl, alkoxy, alkylthio, fluorine, cyano and arylalkyl. Particularly preferred substituents include C₁₋₂₀ alkyl and substituted or unsubstituted aryl, for example phenyl. Optional substituents for the aryl include one or more C₁₋₂₀ alkyl groups.

The extent of conjugation of repeat units of formula (XI) to aryl or heteroaryl groups of adjacent repeat units in the polymer backbone may be controlled by (a) linking the repeat unit through the 3- and/or 6-positions to limit the extent of conjugation across the repeat unit, and/or (b) substituting the repeat unit with one or more substituents R⁸ in or more positions adjacent to the linking positions in order to create a twist with the adjacent repeat unit or units, for example a 2,7-linked fluorene carrying a C₁₋₂₀ alkyl substituent in one or both of the 3- and 6-positions.

The repeat unit of formula (XI) may be a 2,7-linked repeat unit of formula (XIa):

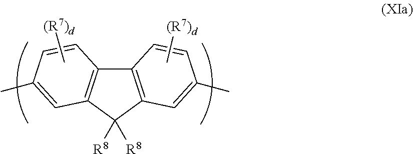

A relatively high degree of conjugation across the repeat unit of formula (XIa) may be provided in the case where each d=0, or where any substituent R₇ is not present at a position adjacent to the linking 2- or 7-positions of formula (XIa).

Conjugation across the repeat unit of formula (XIa) may be limited in the case where at least one d is at least 1, and where at least one substituent R⁷ is present at a position adjacent to the linking 2- or 7-positions of formula (XIa). Optionally, each d is 1 and the 3- and/or 6-position of the repeat unit of formula (XIa) is substituted with a substituent R⁷ to provide a relatively low degree of conjugation across the repeat unit.

The repeat unit of formula (XI) may be a 3,6-linked repeat unit of formula (XIb)

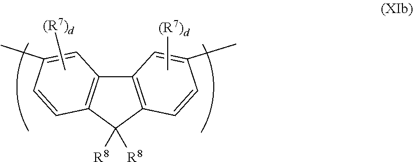

The extent of conjugation across a repeat unit of formula (XIb) may be relatively low as is compared to a corresponding repeat unit of formula (XIa).

Another exemplary arylene repeat unit has formula (VIII):

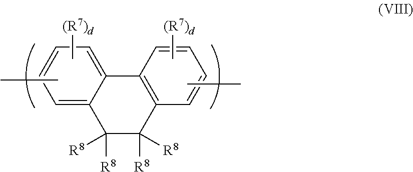

wherein R⁷, R⁸ and d are as described with reference to formulae (IX) and (XI) above. Any of the R⁷ groups may be linked to any other of the R⁷ groups to form a ring. The ring so formed may be unsubstituted or may be substituted with one or more substituents, optionally one or more C₁₋₂₀ alkyl groups.

Repeat units of formula (VIII) may have formula (VIIIa) or (VIIIb):

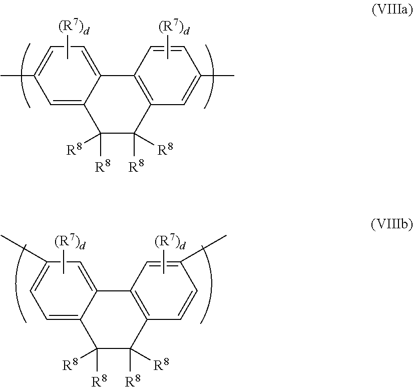

An exemplary repeat unit of formula (VIII) has the following structure, wherein aromatic carbon atoms may each independently be unsubstituted or substituted with a substituent R⁸, and wherein the cyclopentyl groups may each independently be unsubstituted or substituted with one or more substituents, for example one or more C₁₋₂₀ alkyl groups:

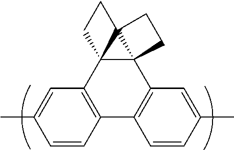

The one or more co-repeat units may include a conjugation-breaking repeat unit, which is a repeat unit that does not provide any conjugation path between repeat units adjacent to the conjugation-breaking repeat unit.

Exemplary conjugation-breaking co-repeat units include co-repeat units of formula (XII):

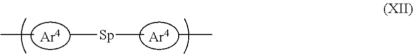

wherein:

Ar⁴ in each occurrence independently represents an aryl or heteroaryl group that may be unsubstituted or substituted with one or more substituents; and

Sp represents a spacer group comprising at least one carbon or silicon atom.

Sp blocks any conjugation path between the two groups Ar⁴. Preferably, the spacer group Sp includes at least one sp³-hybridised carbon atom separating the Ar⁴ groups.

Preferably Ar⁴ is an aryl group and the Ar⁴ groups may be the same or different. More preferably each Ar⁴ is phenyl.

Each Ar⁴ may independently be unsubstituted or may be substituted with 1, 2, 3 or 4 substituents. The one or more substituents may be selected from:
C₁₋₂₀ alkyl wherein one or more non-adjacent C atoms of the alkyl group may be replaced by O, S or COO, C═O, NR⁶ or SiR⁶₂ and one or more H atoms of the C₁₋₂₀ alkyl group may be replaced by F wherein R⁶ is a substituent and is optionally in each occurrence a C₁₋₄₀ hydrocarbyl group, optionally a C₁₋₂₀ alkyl group; and
aryl or heteroaryl, optionally phenyl, that may be unsubstituted or substituted with one or more C₁₋₂₀ alkyl groups.

Preferred substituents of Ar⁴ are C₁₋₂₀ alkyl groups, which may be the same or different in each occurrence.

Exemplary groups Sp include a C₁₋₂₀ alkyl chain wherein one or more non-adjacent C atoms of the chain may be replaced with O, S, —NR⁶—, —SiR⁶2—, —C(═O)— or —COO— and wherein R⁶ in each occurrence is a substituent and is optionally in each occurrence a C₁₋₄₀ hydrocarbyl group, optionally a C₁₋₂₀ alkyl group.

Exemplary repeat units of formula (XII) include the following, wherein R in each occurrence is H or C₁₋₅ alkyl:

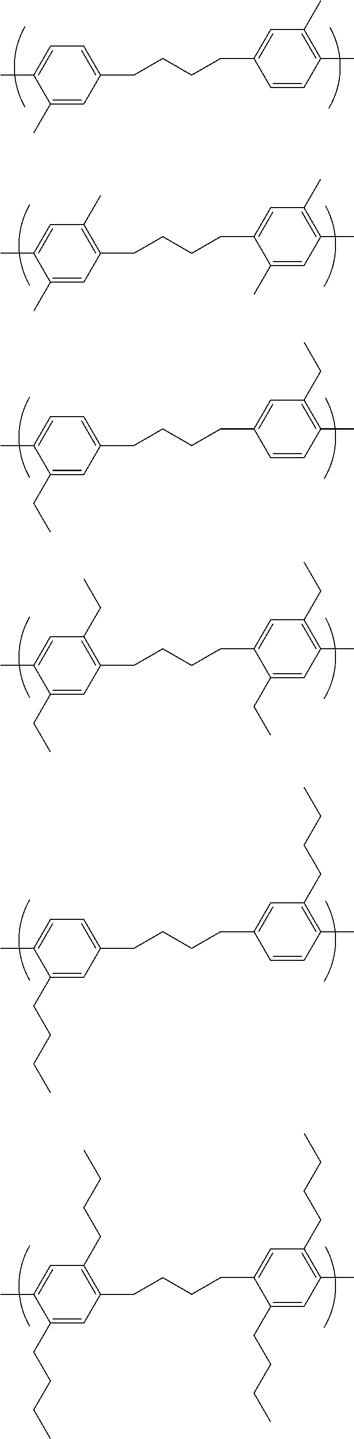

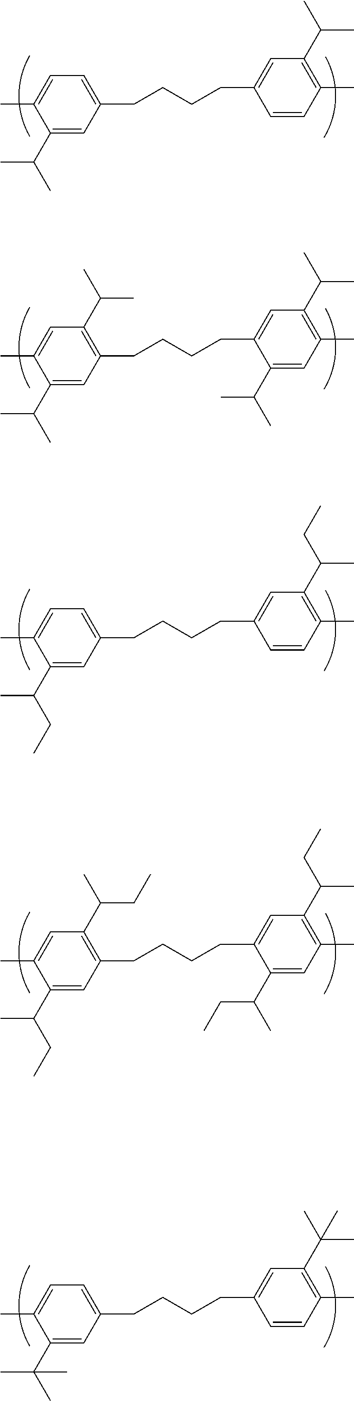

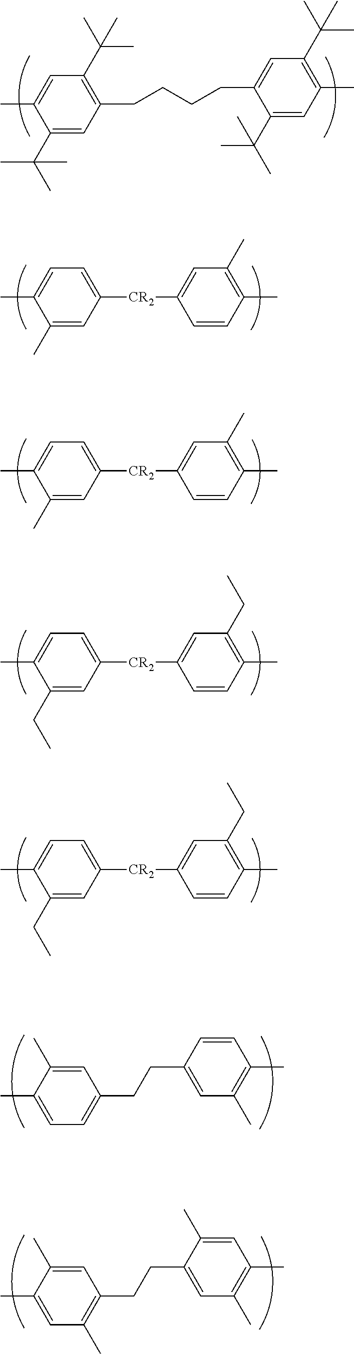

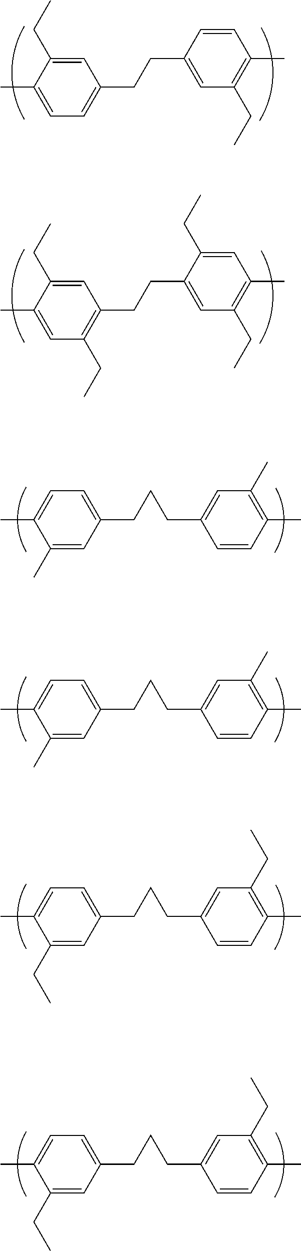

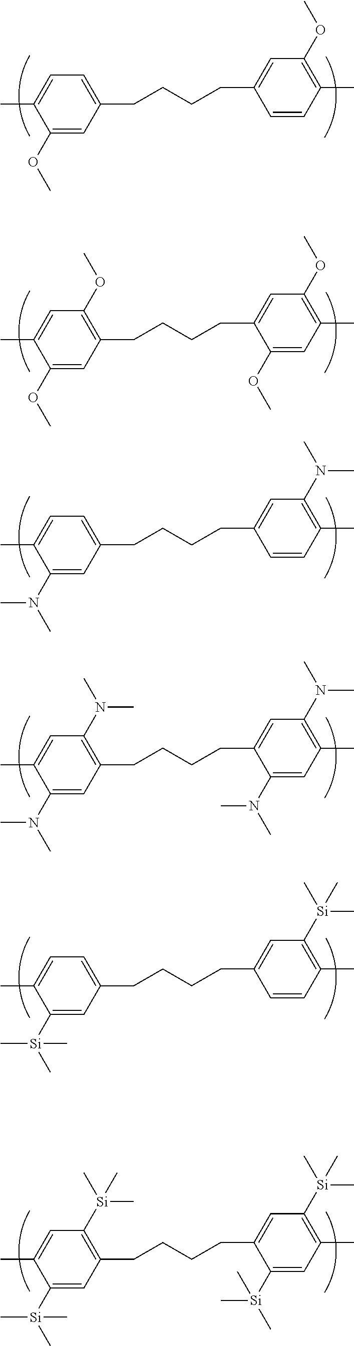

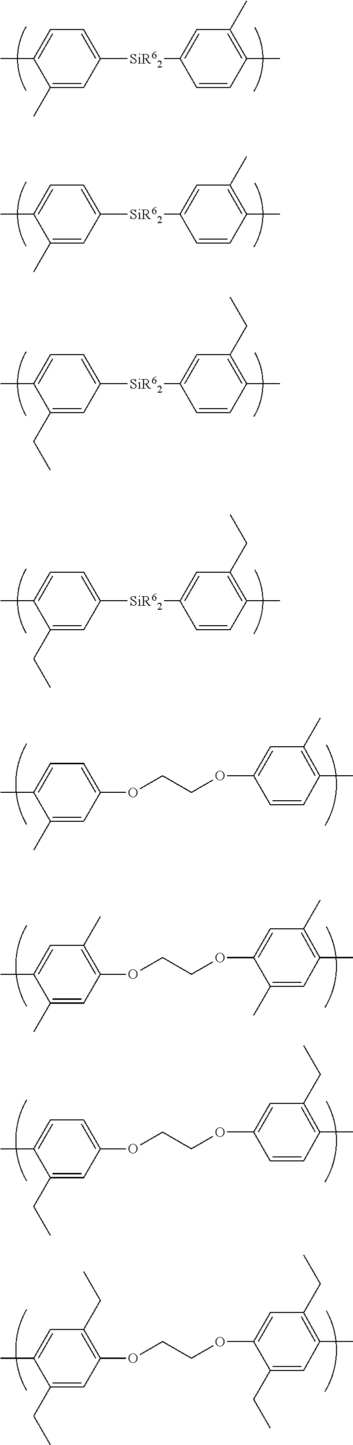

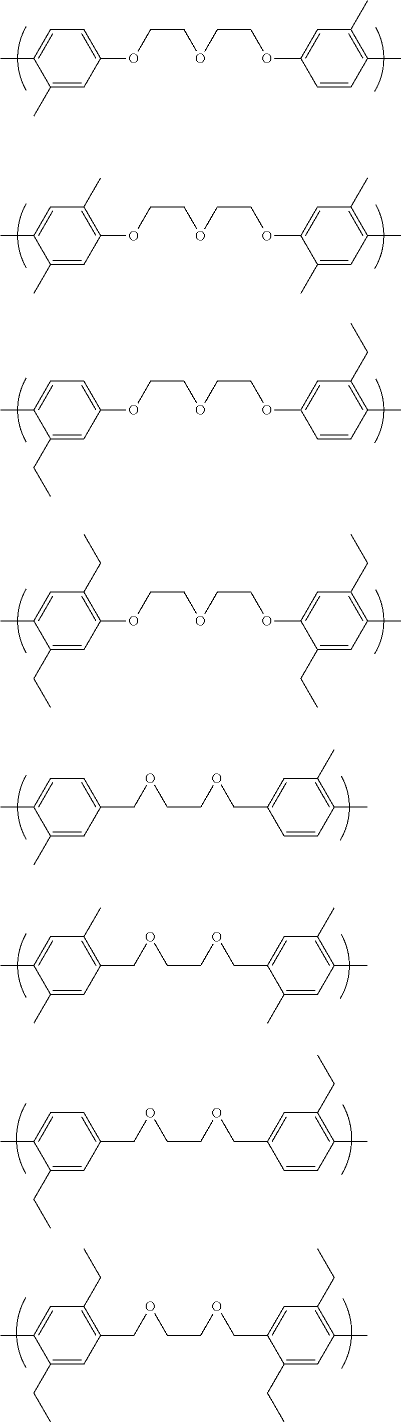

Another exemplary co-repeat unit of formula (XII) has formula (XIIa)

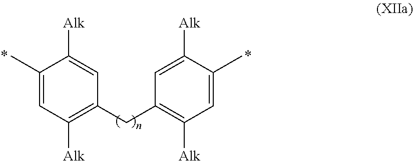

wherein Alk may be independently selected from alkyl, optionally C₁₋₂₀ alkyl and n is at least 1, optionally 1-6, and wherein one or more non-adjacent C atoms may be replaced with optionally substituted aryl or heteroaryl, O, S, substituted N, C═O or —COO—, and one or more H atoms may be replaced with F

Co-repeat units may be formed from corresponding monomers carrying suitable leaving groups.

Applications

Compounds of formula (V), or polymers comprising repeat units of formula (Vr), may be used as a host material in the light-emitting layer of an organic light-emitting device comprising an anode, a cathode and the light-emitting layer between the anode and cathode. The compound of formula (V) may be used in the light-emitting layer in is combination with one or more dopants selected from fluorescent and phosphorescent light-emitting dopants. Preferred phosphorescent light-emitting dopants are phosphorescent transition metal complexes.

### EXAMPLE 1

2,8-diododibenzothiophene was prepared according to the following reaction scheme:

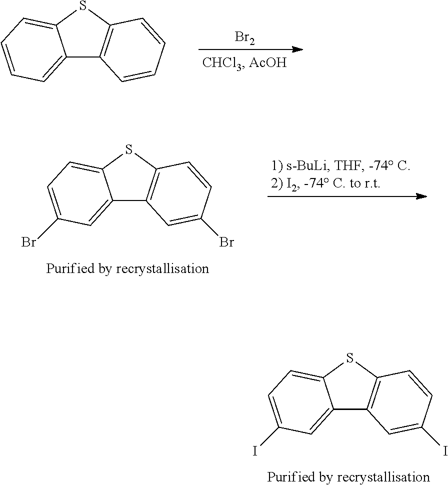 [SMILES: c1ccc2c(c1)sc1ccccc12 ; "Br2" ; Brc1ccc2sc3ccc(Br)cc3c2c1 ; Ic1ccc2sc3ccc(I)cc3c2c1]

Bromine is added to a solution of dibenzothiophene in chloroform and acetic acid. Once the reaction is completed the isolated 2,8-dibromothiophene is purified by a first recrystallisation from n-butyl acetate and a subsequent recrystallisation from toluene (>99.6% purity as measured by HPLC).

s-BuLi is added to a slurry of 2,8-dibromodibenzothiophene in THF at −78° C. Iodine is added to the reaction mixture and the reaction mixture was warned to room temperature overnight. Mixture was cooled down to 0° C. and quenched with water added drop wise. A sample of organic phase was analysed by HPLC: crude 2,8-diiododibenzothiophene=81.21%.

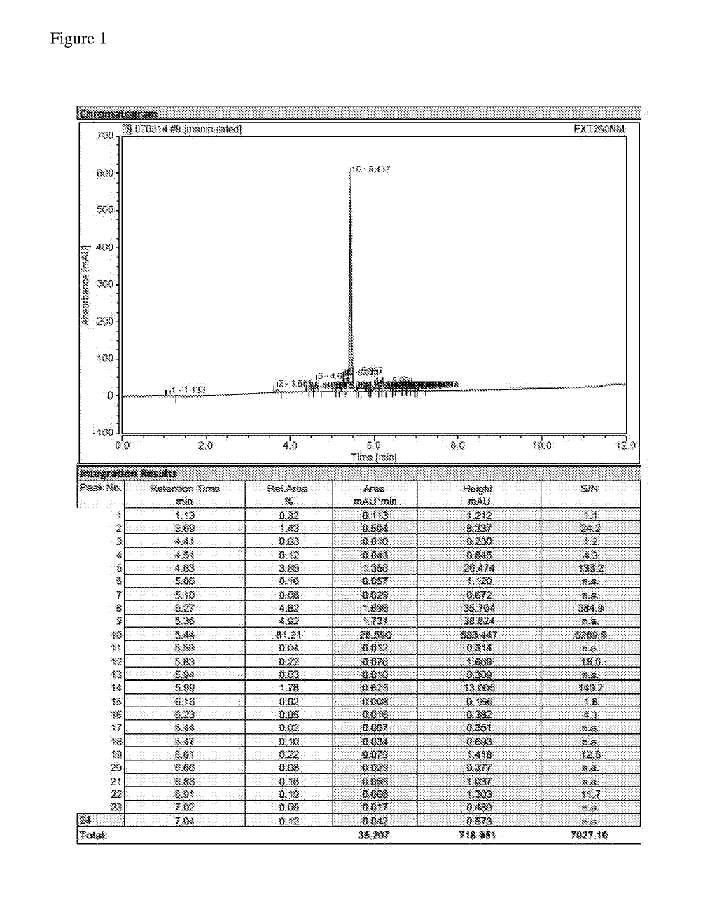

An HPLC chromatogram of a sample of the reaction mixture is shown in FIG. 1. Peak no. 10 at 5.44 min corresponds to 2,8-diiododibenzothiophene and shows this product io represents 81.21% of the crude reaction product.

Sodium bisulfite was added to the reaction mixture as a solid under vigorous stirring until organic phase turned yellow. THF was removed under reduced pressure and the resulting suspension was filtered. Solid was washed 5 times with water, then 3 times with methanol. Solid was dried in vacuum oven at 50° C. for 24 hrs to yield 2,8-diiododibenzothiophene as a white solid (93.11% pure by HPLC, 80% yield).

The isolated crude product can also be recrystallised with toluene to provide 2,8-diiododibenzothiophene (97.35% purity as measured by HPLC).

### DIRECT IODINATION COMPARATIVE EXAMPLE

Dibenzothiophene was iodinated directly with iodine, acetic acid, sulfuric acid and periodic acid in chloroform to obtain 2,8-diiododibezonthiophene. A sample of the organic phase was analysed by HPLC: crude 2,8-diiododibenzothiophene=39.41%

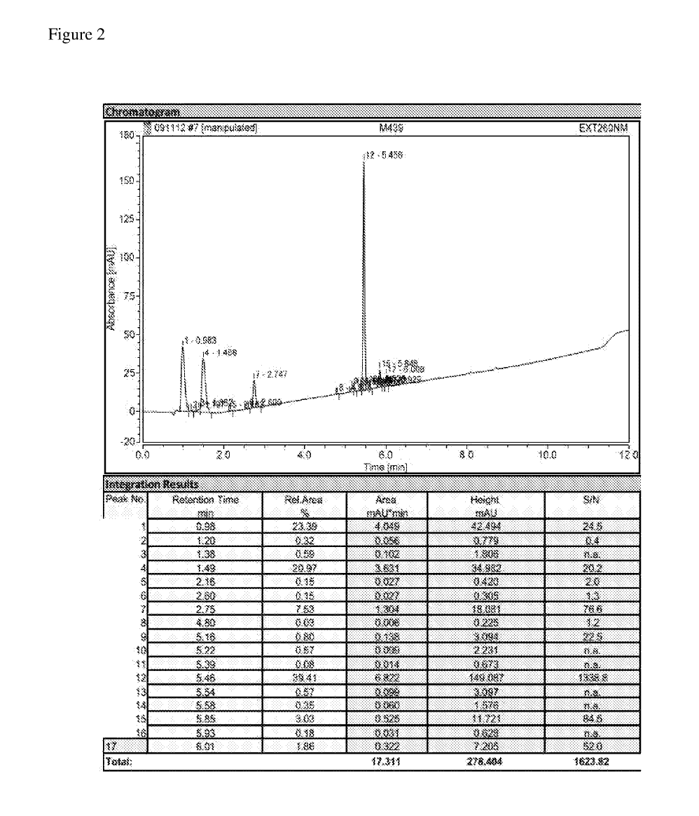

An HPLC chromatogram of a sample of the reaction mixture from direct iodination is shown in FIG. 2. The peak at 5.22 min is starting material, the peaks at 0.98 min, 1.49 min and 2.75 min are polar species coming from oxidised by-product. Peak no. 12 at 5.46 min corresponds to 2,8-diiododibenzothiophene and shows this product represents 39.41% of the crude reaction product.

### MONOMER SYNTHESIS EXAMPLE

A mixture of 2,8-diiododibezothiophene (40.0 g, 91.7 mmol) 3-bromocarbazole (67.7 g, 275.2 mmol), potassium tert-butoxide (35.3 g, 366.9 mmol), copper iodide (34.9 g, 183.5 mmol) and trans-1,2-diaminocyclohexane (4.4 ml, 36.7 mmol) in 830 ml toluene was degassed with nitrogen for 30 minutes. Mixture was heated to 70° C. for 1 hour, then to 90° C. for 24 hours. Heating was increased to 95° C. and stirred for 24 hours.

Mixture was then cooled down to room temperature and filtered through a silica/florisil®/celite plug, eluted with toluene. The filtrate was concentrated under reduced pressure. The resulting solid was dissolved in tetrahydrofuran and adsorbed on isolute® and dry loaded on a silica/florisil® plug. It was eluted with a mixture of toluene:hexane (1:1). Fractions containing product were combined and concentrated under reduced pressure. Resulting solid was recrystallised once from a mixture of toluene and hexane and five times from a mixture of toluene and methanol. Solid was dried in vacuum oven to yield 4.7 g of compound of formula (Va) as a white solid, 95.18% pure by HPLC.

### POLYMERISATION EXAMPLE

Two polymers were prepared by Suzuki polymerisation as described in WO 00/53656 of the following monomers:

| | Composition | | | | | | |
|---|---|---|---|---|---|---|---|
| Poly- | Boronic | | | | | | |
| mer | diesters | Dibromides | Mz | Mw | Mp | Mn | PD |
| Poly- | 50% | 40.41% | 90,000 | 45,000 | 55,000 | 11,000 | 4.23 |
| mer | Boronic | Dibromide 1 | | | | | |
| 1 | ester 1 | 10% | | | | | |
| | | Dibromide 2 | | | | | |
| Poly- | 50% | 50.41% | 103,000 | 54,000 | 65,000 | 12,000 | 4.50 |
| mer | Boronic | Dibromide 1 | | | | | |
| 2 | ester 1 | | | | | | |

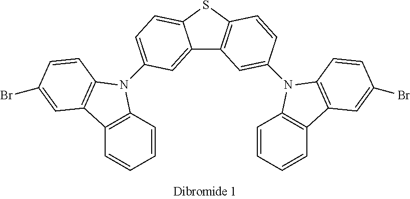 [SMILES: Brc1ccc2c(c1)c1ccccc1n2-c1ccc2sc3ccc(-n4c5ccccc5c5cc(Br)ccc54)cc3c2c1]

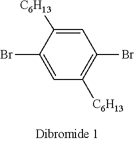

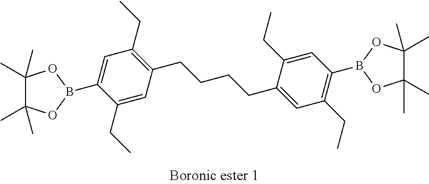

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A process for the preparation of a compound of formula (I): 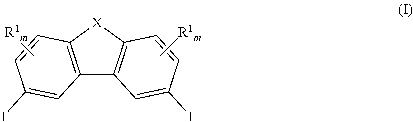
comprising (i) metalation and (ii) iodination of a compound of formula (II): 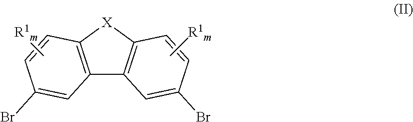
wherein R¹ is the same or different in each occurrence and is a substituent; m independently in each occurrence is 0, 1, 2 or 3; and X independently in each occurrence is NR², PR², —CR²₂—, —SiR²₂, O or S wherein R² is the same or different in each occurrence and is a substituent.

2. The process according to claim 1, wherein X is O.

3. The process according to claim 1, wherein X is S.

4. The process according to claim 1, wherein the metalation is lithiation.

5. The process according to claim 4, wherein the lithiation is carried out using an organolithium.

6. The process according to claim 1, wherein the iodination is carried out using elemental iodine.

7. The process according to claim 1, wherein the compound of formula (I) obtained is purified by recrystallisation.

8. The process according to claim 1, wherein the compound of formula (I) obtained is not purified by chromatography.

9. The process according to claim 1, wherein the compound of formula (II) is prepared by bromination of a compound of formula (III): 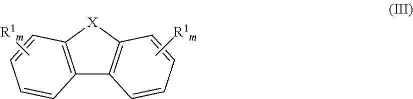
wherein R¹ is the same or different in each occurrence and is a substituent; m independently in each occurrence is 0, 1, 2 or 3; and X independently in each occurrence is NR², PR², —CR²₂—, —SiR²₂, O or S wherein R² is the same or different in each occurrence and is a substituent.

10. The process according to claim 9, wherein the bromination is carried out using elemental bromine.

11. The process according to claim 1, wherein the compound of formula (I) obtained is used in a further step comprising reacting the compound of formula (I) with a compound of formula (IV): 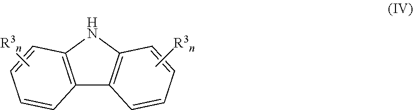
wherein R³ is the same or different in each occurrence and is a substituent; n independently in each occurrence is 0, 1, 2, 3 or 4.

12. The process according to claim 11 wherein the reaction of the compound of formula (I) and the compound of formula (IV) produces a compound of formula (V):

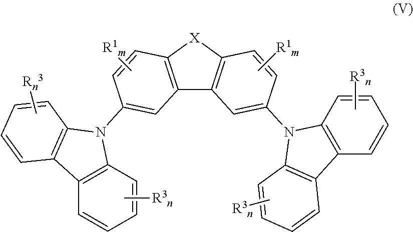

13. The process according to claim 10, wherein at least one n of formula (IV) is at least 1 and at least one R³ is bromine.

14. The process according to claim 10,wherein the compound of formula (IV) is bromocarbazole (IVa):

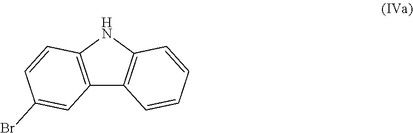 [SMILES: Brc1ccc2[nH]c3ccccc3c2c1]

15. The process according to claim 13 wherein the compound of formula (V) is polymerised to form a polymer comprising a repeat unit of formula (Vr): 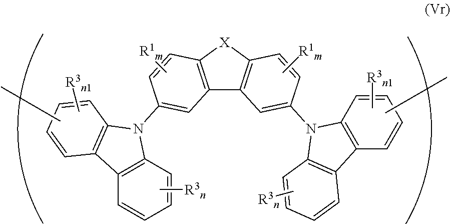
wherein n1 in each occurrence is 0, 1, 2 or 3.

16. A process of forming a compound of formula (V′): 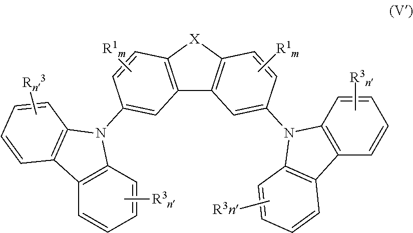
the process comprising the step of reacting a compound of formula (I) and a compound of formula (IV′): 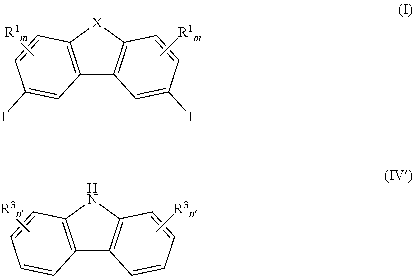
wherein R¹ is the same or different in each occurrence and is a substituent; m independently in each occurrence is 0, 1, 2 or 3; X independently in each occurrence is NR², PR², —CR²₂—, —SiR²₂, O or S wherein R² is the same or different in each occurrence and is a substituent; R³ in each occurrence is a substituent; and n′ in each occurrence is independent 0, 1, 2, 3 or 4, with the proviso that at least one n′ is 1 and at least one R³ is bromine.

17. A partially conjugated co-polymer comprising a repeat unit of formula (Vr) and at least one co-repeat unit: 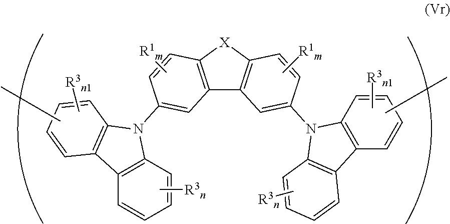
wherein R¹ is the same or different in each occurrence and is a substituent; m independently in each occurrence is 0, 1, 2 or 3; X independently in each occurrence is NR², PR², —CR²₂—, —SiR²₂, O or S wherein R² is the same or different in each occurrence and is a substituent; R³ in each occurrence is a substituent; n in each occurrence is independently 0, 1, 2, 3 or 4; and n1 in each occurrence is independently 0, 1, 2 or 3.

18. A partially conjugated co-polymer wherein at least one co-repeat unit is selected from repeat units of formulae (IX) and (XII) 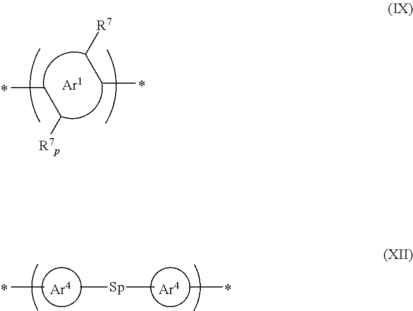
wherein Ar¹ is an arylene group; R⁷ in each occurrence is a substituent adjacent to a linking position of the repeat unit; p is 0 or 1; Ar⁴ in each occurrence independently represents an aryl or heteroaryl group that may be unsubstituted or substituted with one or more substituents; and Sp represents a spacer group comprising at least one carbon or silicon atom.

19. A composition comprising a polymer according to claim 17 and a light-emitting dopant.

20. The composition according to claim 19 wherein the light-emitting dopant is a phosphorescent dopant.

21. An organic light-emitting device comprising an anode, a cathode and a light-emitting layer between the anode and the cathode wherein the light-emitting layer comprises a composition according to claim 19.
